(19) United States
(12) Patent Application Publication
(10) Pub. No.: US 2008/0183207 A1
(43) Pub. Date: Jul. 31, 2008

(54) Tourniquet

(76) Inventor: John N. Horne, Los Alamos, NM (US)

Correspondence Address: William A. Eklund, 126 Northcreek Circle, Walnut Creek, CA, 94598-1315, US

(21) Appl. No.: 11/657,790

(22) Filed: Jan. 25, 2007

(51) Int. Cl. A61B 17/00

(52) U.S. Cl. 606/203

(57) ABSTRACT

A tourniquet having a strap and a pressure applying cam. The cam functions as both a buckle, to allow the strap to be tightened about an injured limb, and also as a pressure applicator that can be selectively positioned and applied to a specific arterial pressure point of the limb. The cam is shaped to have an upwardly extending portion which, when reversed in orientation, extends radially inward toward a limb and can be applied to an arterial pressure point to further control bleeding. In operation, the strap is pre-threaded through the cam for storage and preparation for use. In an emergency the tourniquet is first loosely applied to the injured limb and the strap then tightened and secured to the cam to constrict the limb. The strap is then pulled up and over the cam to rotate it approximately 180 degrees so that the cam is urged against the desired pressure point, while also further shortening the overall length of the tourniquet and thereby further increasing the tension of the tourniquet. The strap is then fastened to itself to secure the tourniquet in place until medical assistance is available.

### BACKGROUND OF THE INVENTION

1. Field of the Invention

The present invention is related to tourniquets for use in emergency medical situations involving trauma to a limb.

2. Description of Related Art Including Information Disclosed Under 37CFR 1.97-1.99

Traditional tourniquets for use in emergency situations involving trauma to a limb rely entirely on constriction of the limb to stem bleeding. However, bleeding from many if not most traumatic limb injuries can be stemmed more effectively through a combination of constriction of the limb and the application of pressure to a point on the limb overlying an underlying artery supplying blood to the limb.

Accordingly, it is an object and purpose of the present invention to provide a tourniquet that constricts a limb while also applying pressure to a desired pressure point on the limb.

It is also an object to provide such a tourniquet that can be applied to a limb easily and quickly in an emergency, and which requires little in the way of fine motor skills or advance training.

It is another object of the present invention to provide a tourniquet that constricts a limb and also applies pressure to a selected pressure point on the limb, while also minimizing tissue damage to the limb during application of the tourniquet and during the time it is applied to the limb.

### SUMMARY OF THE INVENTION

The present invention provides a tourniquet that functions to constrict an injured limb generally, while also applying particular pressure to a selected arterial pressure point on the limb.

The tourniquet includes a strap having first and second ends, and a cam having first and second ends, an upper surface and a lower surface. The first end of the strap is affixed to the first end of the cam. The second end of the cam includes an opening for threading the second end of the strap through the cam, so as to form a loop and to permit the strap to be drawn tightly through the cam to constrict a limb positioned within the loop. The second end of the cam is provided with a locking means for selectively securing the second end of the strap to the second end of the cam, so as to maintain the tourniquet at a desired level of tension around the limb.

As discussed further below, the cam is tipped up and over by approximately 180 degrees, by pulling on the free second end of the secured strap, in order to apply pressure to a desired pressure point. Prior to such tipping, however, the lower surface of the cam constitutes an element of the loop encircling the limb. This point is made here in order to more fully explain the operation of the tourniquet described below.

The upper surface of the cam includes a raised portion extending above the plane defined by the strap and the lower surface of the cam. When the cam is rotated about its first end to a tipped position by pulling the secured second end of the strap up and over the cam, the raised portion of the upper surface of the cam is rotated approximately 180 degrees, so as to extend downwardly and be urged against a desired arterial pressure point of the limb. In being so tipped, the overall length of the tourniquet extending around the limb is also shortened, as the cam is folded back on the strap, such that the constrictive tension of the tourniquet is also increased.

The tourniquet further includes fastening means affixed to the strap for securing the loose second end of the strap in the final position, so as to maintain the cam in the tipped position with the raised portion urged against the arterial pressure point.

In the preferred embodiment an eccentric thumb wheel is journalled to the cam to allow selective securing of the second end of the strap to the cam.

In addition, the preferred embodiment includes a skin shield connected to the first end of the strap and extending alongside the lower surface of the cam, which functions to prevent pinching or bunching of skin as the tourniquet is applied to the limb.

These and other aspects of the present invention will be more apparent upon consideration of the accompanying illustrations and the detailed description of the invention set forth below.

### BRIEF DESCRIPTION OF THE DRAWINGS

The accompanying drawings are incorporated into and constitute a part of this specification and, when taken in combination with the detailed description below, illustrate the operation and construction of the best mode of the invention known to the inventor.

In the Figures:

FIG. 1 is an isometric view of a preferred embodiment of the tourniquet of the present invention;

FIG. 2 is a plan view of the cam of which forms part of the embodiment shown in FIG. 1;

FIG. 3 is a side view of the cam shown in FIG. 2;

FIG. 4 is an end view of the cam shown in FIG. 2, with an associated locking wheel shown;

FIG. 5 is plan view of the cam shown in FIG. 2, with the thumb wheel shown disengaged;

FIG. 6 is a side view of the cam shown in FIG. 2, with the thumb wheel shown engaged with the strap;

FIG. 7 is a plan view of the cam shown in FIG. 2, with the thumb wheel shown in the locking position;

FIG. 8 is a side view of the cam shown in FIG. 2, with the thumb wheel shown in the locking position with a strap secured by it;

FIG. 9 is a side view in partial cross section of the tourniquet of FIG. 1, showing the tourniquet in its first stage of application, with the tourniquet strap pulled tight around a human thigh;

FIG. 10 is a side view in partial cross section of the tourniquet of FIG. 1, showing the tourniquet in its second stage of application, with the strap pulled up and over the cam so as to rotate the cam and apply pressure to an arterial pressure point; and

FIG. 11 is a side view in partial cross section of the tourniquet of FIG. 1, showing the tourniquet in its final stage of application with the strap threaded through a pair of locking D-rings affixed to the strap, to hold the free end of the strap in position.

### DESCRIPTION OF THE PREFERRED EMBODIMENT

Referring to the Figures, a preferred embodiment of a tourniquet 10 made in accordance with the present invention includes a strap 12, a locking cam 14 with adjustable locking wheel 16, and two D-rings 18 and 20.

Briefly, the cam 14 operates as both a buckle and as a manually actuated pressure applicator that operates to apply pressure radially inward to a limb, at a pressure point selected so as to be centered over a major artery in the limb.

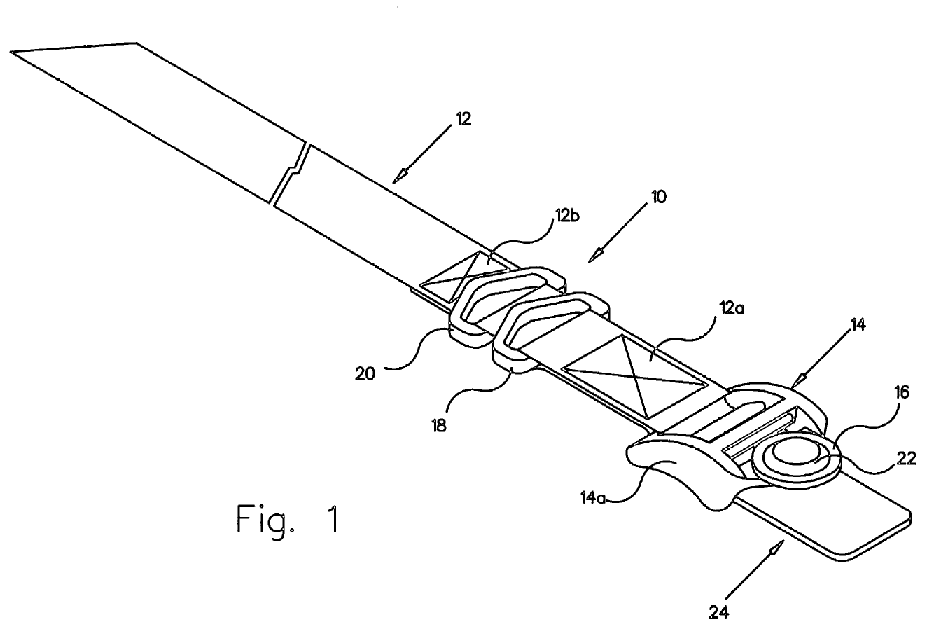

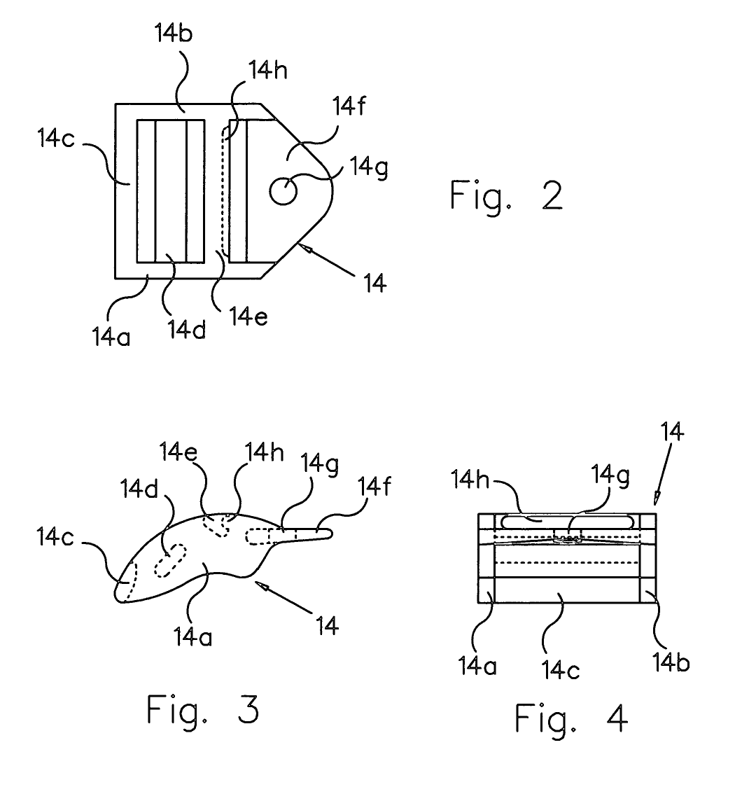

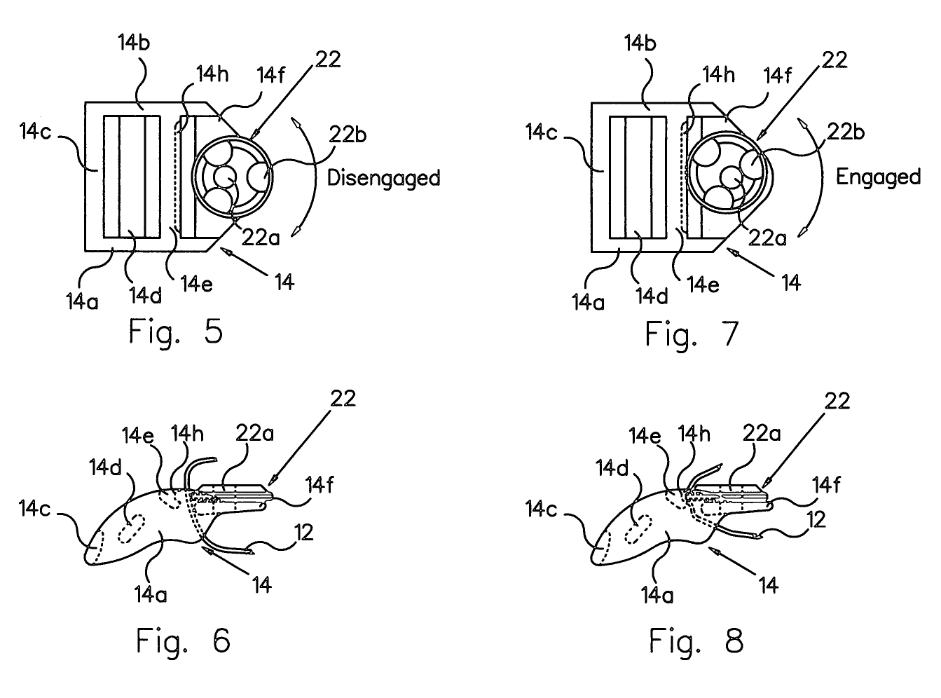

Referring to FIGS. 1 through 6, the cam 14 is preferably formed from a single piece of polymeric structural plastic and includes integral sidewalls 14a and 14b, which are connected by integral end cross bar 14c, middle cross bar 14d and locking cross bar 14e. In addition the cam 14 includes an integral tongue 14f, to which is attached an eccentric locking thumb wheel 22 that is snap-fitted into a hole 14g formed in the tongue 14f.

The eccentric thumb wheel 22 has an integral axle 22a (FIGS. 4 through 8) which is displaced from the geometric center of wheel 22, such that the wheel 22 moves in translation with respect to the cam tongue 14f when the wheel is manually rotated about axle 22a. The eccentric thumb wheel 22 includes thumb recesses 22b to facilitate rotation and adjustment of the position of the wheel 22.

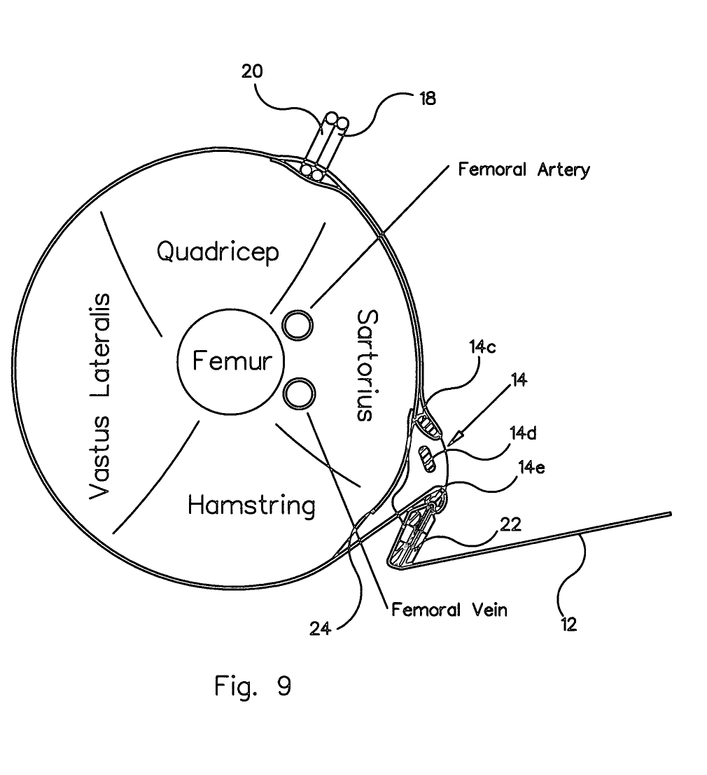

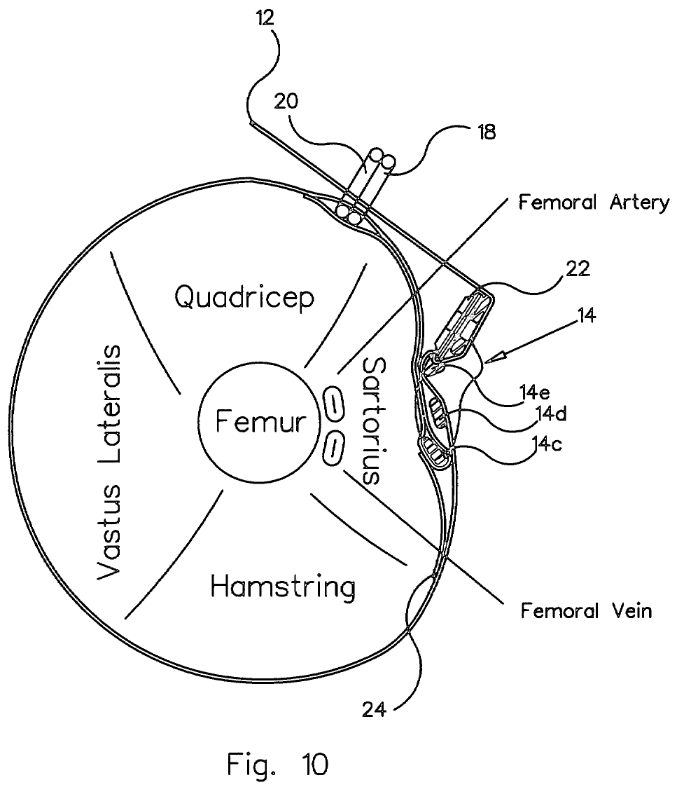

When the strap 12 is threaded through the cam 14 as further described below, the eccentric wheel 22 can be rotated to lock the strap 12 in place, by means of an elongated open recess 14h formed in the locking cross bar 14e. The recess 14h is sized and positioned so as to receive the strap 12 when the strap 12 is forced into the recess 14h by rotation of the thumb wheel 22, as shown in FIG. 9. The thumb wheel 22 can be rotated in either direction, clockwise or counterclockwise, to lock the strap 12 in place or to disengage it, as shown in FIGS. 8 and 9.

A first end of the strap 12 is looped around the end cross bar 14c of the cam 14 and is permanently affixed to the main length of strap 12 by stitching 12a, shown in FIG. 1. From the stitching 12a the main strap 12 extends through the two D-rings 18 and 20. The first end of the strap 12 extends from the stitching 12a around the D-rings 18 and 20 and is affixed to the main length of strap 12 adjacent the D-rings 18 and 20 by additional stitching 12b, as shown in FIG. 1, so that the D-rings 18 and 20 are permanently affixed to the strap 12 adjacent one another in the position shown in FIGS. 1, 9, 10 and 11. In this position the D-rings 18 and 20 may be used in their conventional manner to lock the strap 12 in place after being threaded through them, as described further below.

A skin shield 24 is stitched to the strap 12 at the point where the first end of the strap 12 loops around the end cross bar 14c. The skin shield 24 extends underneath the cam 14 when the cam 14 is in its resting, or initial, position, as shown in FIGS. 1 and 9. The skin shield 24 functions to protect the skin of a limb from being pinched or bunched as the strap 12 is drawn through the cam 14 and tightened, as further described below. The skin shield 24 is formed of sufficiently heavy fabric or polymeric sheeting to resist folding or bunching.

In operation, the tourniquet 10 is prepared for storage and readied for emergency deployment by threading the strap 12 through the cam 14, so that the loosened tourniquet may be simply slipped over a limb and into position in an emergency. More specifically, with the cam 14 in the undeployed position shown in FIG. 1, the strap 12 is formed into a loop and threaded through the cam 14 by first passing it over the skin shield 24, then under the tongue 14f and the locking cross bar 14e, then up over the locking cross bar 14e and back under the cam tongue 14g. The strap 12 is then pulled through the cam 14 sufficiently to prevent the strap 12 from being inadvertently unthreaded from the cam 14 during storage. In this position the tourniquet can be stored indefinitely, with the strap 12 being pre-threaded through the cam 14 so that it is ready to be deployed immediately in an emergency without having to first thread the strap 12 through the cam 14.

In an emergency the tourniquet 10 is first loosened and then slipped over the injured limb, with the cam 14 being positioned adjacent a pressure point overlying a major artery in the limb. The strap 12 is then pulled tight back tightly upon itself so as to constrict the limb and the thumb wheel 22 is then rotated to lock the strap 12 in place, as shown in FIG. 9.

The loose end of the secured strap 12 is then pulled up and over the cam 14, so that the cam 14 is tipped up and rotated approximately 180 degrees about the axis of the end cross bar 14c, to the position shown in FIG. 10. In so rotating the cam 14 pressure is applied to a pressure point on the limb, indicated in FIGS. 9-11 as being generally over the femoral artery and vein of a human thigh.

The side walls 14a and 14b of the cam 14 are shaped arcuately, so that when the cam 14 is tipped up and over, the sidewalls 14a and 14b and the locking cross bar 14e are pressed radially inwardly against the limb, while simultaneously tightening the tourniquet 10 as the strap 12 is drawn tighter around the limb due to shortening of combined length of the strap 12 and the cam 14 in the tipped position.

The arcuate shaping of the cam 14 thus results in the cam 14 applying pressure to a selected point on the limb that overlies a major artery in the limb. The combination of constriction by means of the strap 12, and the application of pressure to a selected arterial pressure point by the shaped cam 14, results in improved control of bleeding from the affected limb.

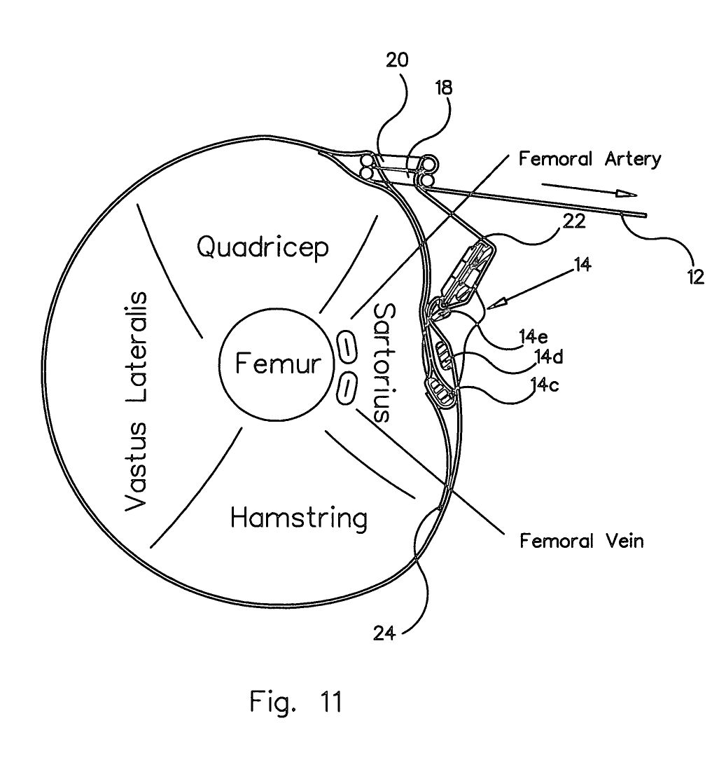

After tipping of the cam 14 while applying sufficient tension to the strap 12 to apply pressure to the pressure point, the strap 12 is first threaded through both D-rings 18 and 20, and then back between the D-rings 18 and 20 and then through D-ring 18, in the conventional manner of using a pair of D-rings to lock a strap in to place. The strap 12 is then pulled tight to lock the strap 12 in place and to also secure the cam 14 in the desired final position and orientation, as shown in FIG. 11.

The tourniquet 10 is quick to apply and does not require fine motor skills to apply or adjust, which skills are typically diminished rapidly in the event of traumatic injuries. The tourniquet utilizes a relatively wide strap, which spreads constrictive pressure over a larger area than conventional tourniquets and thus minimizes tissue damage from the tourniquet itself. Further, the tourniquet does not utilize hook-and-loop fasteners, which can be fouled by blood in an emergency. Perhaps most notably, the tourniquet combines constriction of a limb with direct pressure on an arterial blood supply by means of a single device.

Although the present invention is described herein with reference to a preferred embodiment, it will be understood that various modifications, substitutions and alterations, which may be apparent to one of ordinary skill in the art, may be made without departing from the essence of the present invention. Accordingly, the present invention is described by the following claims.

The embodiments of the invention in which patent protection is claimed are defined as follows:

1. A tourniquet comprising:
a strap having first and second ends;
a cam having first and second ends, an upper surface and a lower surface;
said first end of said strap being affixed to said first end of said cam;
said second end of said cam having an opening for threading said second end of said strap therethrough to form a loop and to permit said strap to be drawn tightly through said cam to constrict a limb positioned within said loop, with said lower surface of said cam constituting an element of said loop;
said second end of said cam including locking means for selectively securing said second end of said strap to said second end of said cam to maintain said loop at a desired level of tension around a limb;
said upper surface of said cam including a raised portion extending above the plane defined by said strap and said lower surface of said cam, whereby said cam may be rotated about said first end of said cam to a tipped position by drawing said second end of said strap up and over said cam, so as to cause said raised portion of said upper surface of said cam to be urged against a desired arterial pressure point of said limb; and
fastening means affixed to said strap for securing said second end of said strap to said strap so as to maintain said cam in said tipped position with said raised portion urged against said pressure point.

2. The tourniquet defined in claim 1 further including a skin shield affixed to said first end of said strap and extending beneath said lower surface of said cam, to prevent pinching as the tourniquet is applied to a limb.

3. The tourniquet defined in claim 1 wherein said locking means includes a rotatable eccentric thumb wheel journalled to said second end of said cam, and wherein said second end of said cam includes a recess against which said thumb wheel urges said strap to secure said strap to said cam.

4. The tourniquet defined in claim 1 wherein said fastening means affixed to said strap includes two D-rings affixed to said strap, whereby said second end of said strap may be threaded through said D-rings to secure said second end of said strap and to maintain said cam in the tipped position.

5. The tourniquet defined in claim 1 wherein said cam includes a pair of side walls connected by a plurality of spaced cross bars, said first end of said strap being affixed to said an end cross bar at said first end of said cam, and wherein said side walls are arcuately shaped upwardly so that when said cam is tipped said side walls and at least one of said cross bars form said raised portion that operates to apply pressure to a selected arterial pressure point on a limb.

6. The tourniquet defined in claim 5 wherein said locking means of said cam includes a tongue extending from said second end of said cam, and wherein said eccentric thumb wheel is journalled to said tongue, and wherein said cam includes a locking cross bar that includes a recess into which said second end of said strap may be urged by rotation of said eccentric thumbwheel, so as to secure said second end of said strap to said cam.

7. The tourniquet defined in claim 6 wherein said locking cross bar and said side walls of said cam constitute said raised portion of said cam operable to apply pressure to an arterial pressure point when said cam is tipped by action of said second end of said strap.

8. A tourniquet comprising:
a strap having first and second ends;
a cam having first and second ends, an upper surface and a lower surface;
said first end of said strap being affixed to said first end of said cam;
said second end of said cam having an opening for threading said second end of said strap therethrough to form a loop and to permit said strap to be drawn tightly through said cam to constrict a limb positioned within said loop, with said lower surface of said cam constituting an element of said loop;
said second end of said cam including locking means for selectively securing said second end of said strap to said second end of said cam to maintain said loop at a desired level of tension around a limb;
said upper surface of said cam including a raised portion extending above the plane defined by said strap and said lower surface of said cam, whereby said cam may be rotated about said first end of said cam to a tipped position by drawing said second end of said strap up and over said cam, so as to cause said raised portion of said upper surface of said cam to be urged against a desired arterial pressure point of the limb; and
two D-rings affixed to said strap and spaced from said first end of said strap for securing said second end of said strap to said strap so as to maintain said cam in said tipped position with said raised portion urged against said pressure point.
